(19) United States
(12) Patent Application Publication
(10) Pub. No.: US 2026/0325641 A1
(43) Pub. Date: Oct. 22, 2026

(54) HOMEOPATHIC COMPOSITION AND METHOD FOR RELIEVING FATIGUE SYMPTOMS

(71) Applicant: BEAURA FOR PARA PHARMACEUTICAL PRODUCTS TRADING L.L.C., Dubai (AE)

(72) Inventor: Yakove Freed, Hod Hasharon (IL)

(73) Assignee: BEAURA FOR PARA PHARMACEUTICAL PRODUCTS TRADING L.L.C., Dubai (AE)

(21) Appl. No.: 19/722,865

(22) Filed: Jun. 28, 2026

Related U.S. Application Data
(63) Continuation-in-part of application No. 18/207,184, filed on Jun. 8, 2023.

(51) Int. Cl. A61K 41/00; A61K 33/24; A61K 33/42; A61K 35/36; A61K 51/00

(52) U.S. Cl. CPC A61K 41/0004; A61K 33/24; A61K 33/42; A61K 35/36; A61K 51/00; A61K 2121/00

(57) ABSTRACT

A homeopathic composition formulated as a single unit dosage form, such as a single tablet, globule, or liquid, comprises a homeopathic carrier and an active homeopathic component. The active homeopathic component consists of five specific ingredients: cadmium sulphuratum, x-ray dilution, phosphoricum acidum, radium bromatum, and carbo animalis. The components are provided within defined potency ranges based on a centesimal dilution scale, preferably featuring cadmium sulphuratum, phosphoricum acidum, radium bromatum, and carbo animalis at a potency of 30C, and x-ray dilution at a potency of 6C. The five active ingredients are present in equal proportions within the final composition. Alternatively, the active component is characterized by comprising an equal volume ratio of each of the five ingredients mixed together prior to its final incorporation into the homeopathic carrier. The composition provides enhanced, unpredictable, and surprising therapeutic efficacy in treating fatigue and exhaustion in oncology patients.

### RELATED APPLICATIONS

This application is a continuation-in-part (CIP) of U.S. patent application Ser. No. 18/207,184, filed on Jun. 8, 2023, the entire contents of which are incorporated herein by reference.

### TECHNICAL FIELD

The present invention relates generally to a novel homeopathic composition and methods for using the same to relieve fatigue symptoms. More particularly, the present invention relates to a specific five-component homeopathic composition structured as a single unit dosage form, such as a single tablet or globule, for alleviating and relieving fatigue symptoms, including radiation therapy fatigue symptoms and cancer treatment-related fatigue symptoms.

### BACKGROUND ART

Fatigue symptoms are now widely recognized as a serious, debilitating medical problem that affects a large segment of the population due to modern lifestyle stressors, chronic illnesses, and medical interventions. In particular, cancer treatment-related fatigue symptoms—including exhaustion induced by chemotherapy, radiation therapy, immunotherapy, or surgery—constitute a severe clinical challenge. This form of fatigue is uniquely profound, persistent, and typically unalleviated by rest, drastically reducing a patient's quality of life, appetite, and body weight.

Conventional allopathic pharmacology offers limited, often unsatisfactory solutions for cancer treatment-related fatigue, frequently introducing undesirable side effects or drug-drug interactions. Accordingly, there is an urgent, unfulfilled medical need for a safe, non-toxic, and effective therapeutic intervention capable of relieving these fatigue symptoms without interfering with primary oncological treatments.

In classical homeopathy, a composition is understood to be an indivisible, synergistic entity. Unlike conventional allopathic pharmacology where chemical effects may be merely linear or additive, a homeopathic composition cannot be modified by adding or deleting remedies without completely altering, neutralizing, or destroying its specific energetic and therapeutic profile. The specific, exclusive combination of the five ingredients defined herein structured into a single unit dosage form creates a unique, unpredictable synergistic effect for relieving cancer treatment-related fatigue that cannot be achieved by prior art formulas individually, nor by adding further active remedies to the matrix.

### DESCRIPTION OF THE DRAWINGS

The intention of the drawings attached to the application is not to limit the scope of the invention and its application. The drawings are intended only to illustrate the invention and they constitute only one of its many possible implementations.

FIG. 1 illustrates a subject (100) experiencing fatigue symptoms in need of treatment.

FIG. 2 illustrates the active homeopathic component (200B) structured as a single unit dosage form and its constituent ingredients (201, 202, 203, 204, 205).

FIG. 3 illustrates the homeopathic composition 200.

### THE INVENTION

Homeopathy is an established, highly specialized discipline of complementary medicine in which practitioners treat patients using micro-diluted potentized substances. These ingredients are prepared through successive, standardized steps of dilution and succussion (vigorous shaking), which enable the body's natural defense, adaptive, and homeostatic healing mechanisms. Homeopathic remedies are recognized and regulated under U.S. federal law, specifically the Federal Food, Drug, and Cosmetic Act of 1938, which mandates that homeopathic remedies be manufactured and regulated in accordance with the Homeopathic Pharmacopoeia of the United States (HPUS).

### The Synergistic Core Product Formulation

A principal object of the present invention is to provide a homeopathic composition (200) for relieving fatigue symptoms (including radiation fatigue symptoms and cancer treatment-related fatigue symptoms) in a subject (100) in need thereof.

To achieve the precise, non-obvious synergistic therapeutic profile required to treat post-cancer exhaustion, the active core of the composition is strictly limited.

In a preferred embodiment, the invention relates to a homeopathic composition formulated as a single tablet or globule, comprising a pharmaceutically acceptable homeopathic carrier and an active homeopathic component, wherein said active homeopathic component consists of: Cadmium sulphuratum (201); X-ray dilution (202); Phosphoricum acidum (203); Radium bromatum (204); and Carbo animalis (205).

In an alternative and/or a specific embodiment, the homeopathic composition is formulated as a single tablet or globule consisting essentially of: (a) cadmium sulphuratum; (b) x-ray dilution; (c) phosphoricum acidum; (d) radium bromatum; (e) carbo animalis; and (f) a pharmaceutically acceptable homeopathic carrier selected from sucrose, lactose, or a combination thereof.

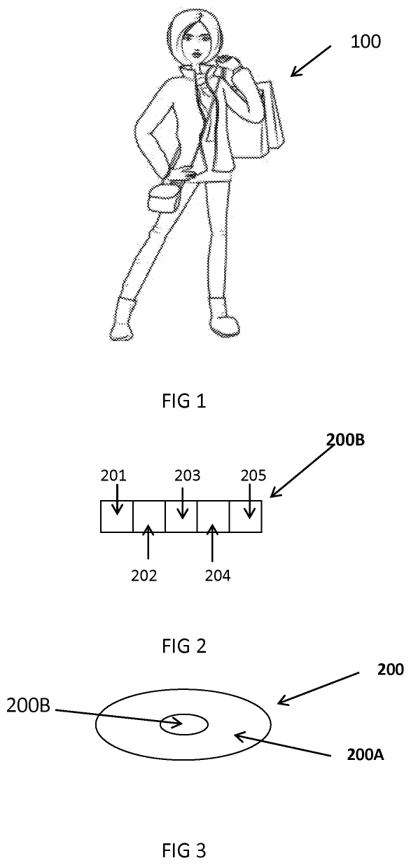

FIG. 1 illustrates a subject (100), and FIG. 2 illustrates the active homeopathic component (200B) and its specific ingredients. The individual homeopathic ingredients utilized herein are known within classical repertories to possess distinct pathogenic profiles:
Cadmium sulphuratum is indicated for extreme prostration, progressive weakness, and gastric irritability.
Phosphoricum acidum is historically indicated for mental and physical apathy, nervous exhaustion, and severe vital fluid depletion.
Carbo animalis is indicated for profound constitutional debility, cachexia (severe weight loss and muscle wasting), and lack of vital heat.
Radium bromatum and X-ray dilution are highly specialized potentized preparations. The X-ray dilution is a homeopathic compound prepared by exposing a designated vial of alcohol to X-ray radiation under standardized conditions. Both remedies exhibit specific affinity for tissue damage, cellular depletion, and profound lethargy directly following exposure to ionizing radiation or radioactive compounds.

### Standardized Dilution Scales and Potency Ranges

The English letter “CH” (or interchangeably “C”) following a numeral beside the name of a homeopathic ingredient denotes the Centesimal scale, indicating that the ingredient has underwent a sequential series of dilutions on a scale of 1:100, accompanied by standardized succussion at each stage. The numeral denotes the exact number of successive centesimal dilution cycles performed.

For example, 30 C means that the starting active mother tinctures or crude substance was diluted to a 1/100 concentration sequentially thirty times, resulting in a theoretical dilution factor of 10⁻⁶¹.

To achieve optimal, reliable, and clinically repeatable relief from post-treatment cancer exhaustion, the ingredients of the active homeopathic component are prepared within specified potency windows:
The cadmium sulphuratum should be diluted to a potency of not less than 12 C and not more than 200 C. In an optimal embodiment, it is potentized to exactly 30 C.
The x-ray dilution should be diluted to a potency of not less than 3 C and not more than 30 C. In an optimal embodiment, it is potentized to exactly 6 C.
The phosphoricum acidum should be diluted to a potency of not less than 12 C and not more than 200 C. In an optimal embodiment, it is potentized to exactly 30 C.
The radium bromatum should be diluted to a potency of not less than 12 C and not more than 200 C. In an optimal embodiment, it is potentized to exactly 30 C.
The carbo animalis should be diluted to a potency of not less than 12 C and not more than 200 C. In an optimal embodiment, it is potentized to exactly 30 C.

### Physical Delivery Format and Ratios

The composition of the present invention is structurally integrated into a single, unified unit dosage form. The homeopathic composition can be solid (such as molded tablets or compressed globule pellets) engineered to be dissolved sublingually in the oral cavity of the subject, or liquid (such as potentized drops) to be administered orally. The weight or volume ratios of the constituent active ingredients within the composition may be varied. However, to guarantee an optimal, balanced therapeutic response, the composition comprises an equal ratio or proportion of each of the five ingredients.

For example, each of the five distinct potentized remedies constitutes one-fifth (20%) of the total active liquid mixture sprayed onto or incorporated into the final tablet or globule base. In an embodiment, the five active homeopathic remedies of the present invention are combined and incorporated into the single unit dosage form (e.g., the single tablet or globule) via a standardized impregnation process. Specifically, separate liquid potentized preparations of cadmium sulphuratum, x-ray dilution, phosphoricum acidum, radium bromatum, and carbo animalis are provided in their respective designated potencies. Prior to application onto the solid carrier matrix, a unified liquid remedy mixture is prepared by combining these five distinct liquid preparations in equal volume parts (an equal volume ratio of 1:1:1:1:1). This unified, balanced liquid mixture is subsequently utilized to impregnate, saturate, or spray a non-medicated, pharmaceutically acceptable solid carrier matrix, such as neutral sucrose or lactose globules or compressed tablets. Following the impregnation step, the medicated solid carrier is dried under controlled conditions to remove residual moisture, thereby locking the integrated active homeopathic energetic signature uniformly into each single tablet or globule. This method ensures that each individual unit dosage form delivers an identical, precisely balanced therapeutic dose of the five-ingredient synergistic core. As used in this disclosure, ‘equal proportions’ in the active homeopathic component may refer to equal proportions by weight (w/w), equal proportions by volume (v/v), equal molar proportions, or equal concentration ratios. In a preferred embodiment, the five active components are present in a substantially 1:1:1:1:1 ratio relative to one another. Alternatively: Prior to incorporation into the single tablet or globule, the active homeopathic components are blended in an equal volume ratio (e.g., 1:1:1:1:1). This combined active liquid component is then incorporated into the homeopathic carrier (such as lactose, sucrose, or xylitol) by means of spraying, triturating, geometric dilution, or fluid-bed impregnation. Throughout this specification and in the claims, the terms ‘equal proportions,’ ‘equal volume ratio,’ or ‘same ratio’ are understood to encompass slight manufacturing variances. Specifically, ‘equal’ includes ratios within a margin of ±5%, preferably ±2%, and most preferably ±0.5% of a strict 1:1:1:1:1 equilibrium. The final single tablet or globule is characterized as the product resulting from the impregnation of a carrier with an active component consisting of an equal volume blend of ingredients (a) through (e). Even if subsequent processing, drying, or compression alters the absolute mass of individual components, the structural and therapeutic identity of the active component is defined by its equal-ratio liquid precursor.

### Methods of Medical Administration

Another object of the present invention is to provide a method for relieving fatigue symptoms (including radiation fatigue symptoms and cancer treatment-related fatigue symptoms) in a subject in need thereof. The method comprises orally administering to the subject a therapeutically effective amount of the five-component homeopathic composition described above, wherein each individual administration event comprises taking a single tablet or globule.

The single tablet or globule is preferably administered orally or sublingually at a frequency, depending on the severity of the patient's exhaustion. The method of treatment described herein may be performed utilizing any of the specific potency ranges or preferred single-potency embodiments (such as the 30 C/6 C matrix) disclosed herein.

### Pharmacological Criteria, Synergy, and the “Teaching Away” Doctrine

The claimed five-ingredient homeopathic composition represents a specific, non-obvious advancement in the art that directly overcomes technical prejudices and “teaching away” warnings established in foundational pharmacological literature.

### The Problem of Component Substitution and Complex Mixtures

In conventional allopathic pharmacology, chemical substances interact primarily via mass-action laws and linear receptor pathways, where effects are frequently additive or predictable. Conversely, in the specialized field of homeopathic pharmacology, a multi-component composition is not a mere aggregation of individual parts. Rather, the holistic interaction between all specific components creates a unique, indivisible medicinal “totality.”

Consequently, removing a specific component from a known multi-component formula and substituting it with new fifth element is not a matter of routine optimization or obvious experimentation for a Person Having Ordinary Skill in the Art (PHOSITA). A PHOSITA would recognize that swapping out a core element fundamentally alters, destabilizes, or invalides the synergistic profile of the entire remedy.

For instance, while a formulation comprising Cadmium sulphuratum, X-ray, Phosphoricum acidum, Radium bromatum, and Carcinosinum burnett represents one distinct profile (as seen in Dolev et al., where the present inventor was a primary researcher), the present invention represents a distinct, non-obvious departure from that formulation. Removing Carcinosinum burnett and introducing Carbo animalis creates an entirely separate, non-equivalent clinical totality specifically calibrated for post-oncology exhaustion. The prior art provides no direction, lead, or motivation as to why Carbo animalis should be selected to replace a distinct component, or why such a specific modification would enhance clinical efficacy.

It must be emphasized that the prior art disclosure of Dolev et al. relies on the successive administration of five separate remedies given sequentially over time to the patient. Conversely, the composition of the present invention is a single, unified composition structured as one single tablet or globule, which consists exclusively of five specific active ingredients in a fundamentally different architectural form.

Generally, in homeopathic formulation, taking a multi-component remedy, removing a single component therefrom, and substituting it with an entirely different ingredient creates a completely distinct composition that possesses therapeutic effects entirely different from the sum of its baseline components. This principle of non-linear structural unpredictability applies to both homeopathic formulations and conventional allopathic medications alike.

Furthermore, a critical distinction must be maintained between a crude botanical extract (a herbal phytotherapeutic agent) and a potentized homeopathic remedy. The mere fact that a crude plant extract is known to be beneficial for a specific physical symptom does not teach, suggest, or imply that a homeopathic potentization of that same substance will possess the same therapeutic indication.

In pharmacological science, crude herbal medicine and homeopathic potentization represent fundamentally divergent fields of art operating under entirely separate bio-logical mechanisms. Crude extracts rely on mass-action chemical concentrations to induce a response, whereas homeopathic remedies rely on sequential dilution and succussion to preserve informational and energetic cellular signatures. Consequently, it is entirely unacceptable and contrary to established scientific principles to extrapolate efficacy or draw obviousness conclusions between these two distinct disciplines.

### Foundational “Teaching Away” in Homeopathic Science

Furthermore, a PHOSITA attempting to combine multiple medicinal remedies into a single dosage form would be actively discouraged and taught away from doing so by the governing, definitive literature of the field. Samuel Hahnemann, the founder of homeopathic pharmacology, explicitly warned against the deep unpredictability and potential inhibitory interference of combining complex medicinal substances.

In the Organon of Medicine (6th Edition, translated by William Boericke), which serves as the foundational pharmacological text in this discipline, Aphorism § 272 and its accompanying notes state:
“It is absolutely not allowed in Homoeopathy . . . to give the patient at one time two different medicinal substances.”

Aphorisms § 273-274 further outline that multiple medicinal substances administered simultaneously do not produce a simple, double, or predictable linear effect, but rather a completely new, unmapped action that may neutralize or harm the patient's vital force. Because the established literature explicitly warns that multi-ingredient combi-nations create highly unpredictable, potentially inhibitory actions, a PHOSITA would possess an inherent technical prejudice against formulating complex mixtures.

The present invention overcomes this long-standing technical prejudice. By utilizing a highly specific, closed five-ingredient matrix (consisting of components a, b, c, d, and e) structured as a single tablet or globule, the present invention captures an unexpected, stable, and highly effective therapeutic synergy that defies the traditional negative expectations detailed in the art.

### Distinguishing Over Overbroad “Laundry List” Disclosures

Generic prior art references that disclose broad, water-soluble carrier powders (such as cannabinoid powders or general surfactant matrices) often include exhaustive, overbroad lists of potential optional additives. These disclosures frequently encompass hundreds of essential oils, thousands of herbal extracts, and over 1,500 listed homeopathic remedies.

Under established patent principles, such a “laundry list” disclosure fails to render a specific combination obvious when it requires a PHOSITA to blindly pick, choose, and combine disparate elements from thousands of options without a specific pharmacological rationale, value-added data, or clear “direction or lead” pointing to the specific five-element combination (see In re Baird, 16 F. 3d 380). The focus of such references is fundamentally divorced from the specialized discipline of homeopathic potentization, synergistic component matching, and cell-memory preservation. The present invention's precise five-ingredient selection is not a matter of obvious, random mixing, but a specific, non-obvious discovery.

### Clinical Examples and Experimental Results

The specific five-ingredient homeopathic composition of the present invention was manufactured by mixing equal liquid volume parts of Cadmium sulphuratum (30 C), X-ray dilution (6 C), Phosphoricum acidum (30 C), Radium bromatum (30 C), and Carbo animalis (30 C). This active component mixture was then uniformly impregnated onto standard lactose/sucrose globules to form single, unified unit-dosage tablets.

The composition was administered to a clinical cohort of patients undergoing active radiation therapy and chemotherapy regimens who were suffering from severe, un-remitting cancer treatment-related fatigue symptoms. The tablets were administered orally/sublingually at a dosing frequency of three to five times daily over the course of their oncological treatment cycles.

The clinical results demonstrated an immediate and unexpected therapeutic response that was not achievable by administering the individual ingredients or sub-combinations thereof. Patients treated with the single-tablet combination demonstrated: (1) A rapid, statistically significant reduction and relief in subjective and objective fatigue scores. (2) A sharp, unexpected increase in metabolic appetite. And (3) A reversal of cachexia, leading to a stabilization and marked increase in patient body weight.

No adverse events, side effects, or inhibitory interference with the primary oncological treatments were observed, confirming that the precise closed matrix of the five specified ingredients creates an unpredictable, safe, and highly effective clinical synergy.

The present invention relates to a homeopathic composition formulated as a single dosage form, such as a single tablet, globule, or liquid, consisting of: cadmium sulphuratum (30 C), X-ray dilution (6 C), phosphoricum acidum (30 C), radium bromatum (30 C), and carbo animalis (30 C), present in equal proportions.

Prior Art reference Dolev et al., Integrative Cancer Therapies, 2021 (hereinafter “Dolev”) discloses a homeopathic mixture comprising five separate ingredients, namely: cadmium sulphuratum (30 C), X-ray dilution (6 C), phosphoricum acidum (30 C), radium bromatum (30 C), and carcinosinum burnett (30 C). The inventor conducted clinical observations within his clinic across a wide spectrum of oncology patients (and others) suffering from fatigue and exhaustion. A cohort of these patients was administered the five separate remedies disclosed by Dolev, while another cohort was administered the formulation of the present invention, wherein the five specific active components are combined and consolidated into a single unit dosage form.

The clinical outcomes demonstrated surprising and unexpected therapeutic efficacy. While the patients treated with the separate remedies of Dolev exhibited only minor to imperceptible improvements in their level of fatigue and exhaustion, the patients who received the unitary composition of the present invention demonstrated a highly significant, pronounced, and visible improvement.

It is emphasized that these clinical results represent a surprising effect that is entirely non-obvious. A person skilled in the art of homeopathy, if presented with the modification of removing a single component from the Dolev mixture and replacing it with carbo animalis (30 C) to arrive at the present composition, would be entirely unable to predict or anticipate the resulting therapeutic outcome. Within the field of homeopathy, any alteration, substitution, or modification of an individual component within a multi-remedy composition results in an unpredictable physiological response that cannot be determined a priori.

### Composition and a Method

The present invention refers to the homeopathic composition (200) that is formulated as a single tablet, globule or liquid, The composition comprising a homeopathic carrier (200A) and an active homeopathic component (200B). The active homeopathic component consists of: (a) cadmium sulphuratum; (b) x-ray dilution; (c) phosphoricum acidum; (d) radium bromatum; and (e) carbo animalis. FIG. 3 illustrates the composition 200.

The cadmium sulphuratum may be at a potency of 12 C to 200 C; (b) the x-ray dilution may be at a potency of 3 C to 30 C; (c) the phosphoricum acidum may be at a potency of 12 C to 200 C; (d) the radium bromatum may be at a potency of 12 C to 200 C; and (e) the carbo animalis may be at a potency of 12 C to 200 C. Wherein “C” denotes a centesimal dilution scale of 1:100. The cadmium sulphuratum may be at a potency of 30 C; (b) the x-ray dilution may be at a potency of 6 C; (c) the phosphoricum acidum may be at a potency of 30 C; (d) the radium bromatum may be at a potency of 30 C; and (e) the carbo animalis may be at a potency of 30 C. The cadmium sulphuratum, said x-ray dilution, the phosphoricum acidum, the radium bromatum, and the carbo animalis are may be present in the active homeopathic component in equal proportions or comprises an equal volume ratio of each of them prior to their incorporation into the single tablet or globule. For clarity, the present invention refers to the method for relieving cancer treatment-related fatigue symptoms in a subject in need thereof, comprising administering to the subject the homeopathic composition.

What we claim is:

1. A homeopathic composition formulated as a single tablet, globule or liquid, comprising a homeopathic carrier and an active homeopathic component, wherein said active homeopathic component consists of: (a) cadmium sulphuratum; (b) x-ray dilution; (c) phosphoricum acidum; (d) radium bromatum; and (e) carbo animalis.

2. The homeopathic composition of claim 1, wherein: (a) said cadmium sulphuratum is at a potency of 12 C to 200 C; (b) said x-ray dilution is at a potency of 3 C to 30 C; (c) said phosphoricum acidum is at a potency of 12 C to 200 C; (d) said radium bromatum is at a potency of 12 C to 200 C; and (e) said carbo animalis is at a potency of 12 C to 200 C; wherein “C” denotes a centesimal dilution scale of 1:100.

3. The homeopathic composition of claim 2, wherein: (a) said cadmium sulphuratum is at a potency of 30 C; (b) said x-ray dilution is at a potency of 6 C; (c) said phosphoricum acidum is at a potency of 30 C; (d) said radium bromatum is at a potency of 30 C; and (e) said carbo animalis is at a potency of 30 C.

4. The homeopathic composition of claim 1, wherein said cadmium sulphuratum, said x-ray dilution, said phosphoricum acidum, said radium bromatum, and said carbo animalis are present in the active homeopathic component in equal proportions.

5. The homeopathic composition of claim 1, wherein the active homeopathic component comprises an equal volume ratio of each of said cadmium sulphuratum, said x-ray dilution, said phosphoricum acidum, said radium bromatum, and said carbo animalis prior to its incorporation into the single tablet or globule.
